(19) United States
(12) Patent Application Publication
(10) Pub. No.: US 2023/0399368 A1
(43) Pub. Date: Dec. 14, 2023

(54) CARRIER PEPTIDE FRAGMENT AND USE THEREOF

(71) Applicant: TOAGOSEI CO., LTD., Tokyo (JP)

(72) Inventors: Nahoko Baileykobayashi, Ibaraki-ken (JP); Tetsuhiko Yoshida, Ibaraki-ken (JP); Mikio Niwa, Ibaraki-ken (JP)

(21) Appl. No.: 18/249,988

(22) PCT Filed: Oct. 14, 2021
(86) PCT No.: PCT/JP2021/037981

§ 371 Date: Apr. 21, 2023

(30) Foreign Application Priority Data
Oct. 23, 2020 (JP) 2020-177944

(51) Int. Cl. C07K 14/47; C07K 19/00

(52) U.S. Cl. CPC C07K 14/47; C07K 19/00

(57) ABSTRACT

The present disclosure provides art that can efficiently introduce a foreign substance of interest from the outside of a eukaryotic cell into at least the cytoplasm of the cell. The herein disclosed method is a method for introducing a foreign substance of interest from the outside of a eukaryotic cell into at least the cytoplasm of the cell, wherein the method comprises: (1) a step of preparing a foreign substance introduction construct that has a carrier peptide fragment comprising MAPRGFSCLLLLTSEIDLPVKRRA (SEQ ID NO: 1) and the foreign substance of interest bonded to the N-terminal side and/or the C-terminal side of the carrier peptide fragment; (2) a step of supplying the foreign substance introduction construct into a sample that contains a target eukaryotic cell; and (3) a step of incubating the sample into which the foreign substance introduction construct has been supplied, to thereby introduce the construct into the eukaryotic cell in the sample.

### TECHNICAL FIELD

The present invention relates to a method for introducing (transferring) a foreign substance from the outside of a eukaryotic cell into the interior of the cell, and further relates to a carrier peptide fragment used in this method. The present application claims priority based on Japanese Patent Application No. 2020-177944 filed Oct. 23, 2020, the contents of which are incorporated in their entirety in the present Description by reference.

### BACKGROUND ART

Foreign substances and particularly bioactive substances, e.g., polypeptides, have been introduced into the cells (eukaryotic cells) of, e.g., humans and other mammals, in order to change the characteristics of the cells (as well as tissues and organs composed of the cells) and/or to enhance or improve the function of the cells.

For example, Patent Literature 1 discloses a cell-penetrating carrier peptide for the introduction of a foreign substance, e.g., a polypeptide or DNA, into a cell. This patent literature teaches that a bioactive substance, e.g., a polypeptide or DNA, can be introduced at high efficiencies into a cell by using a carrier peptide conjugate in which a cell-penetrating carrier peptide is linked to, e.g., a heterologous polypeptide, DNA, and so forth.

For example, Patent Literature 2 discloses a foreign substance introduction construct that contains a foreign substance of interest and the amino acid sequence described in SEQ ID NO: 2, which is described in Non Patent Literature 1 and is known as the nucleolar localization signal (also abbreviated as “NoLS” in the following) of LIM kinase 2. LIM kinase 2 is a type of protein kinase that is present in human endothelial cells and participates in intracellular signaling. Because this amino acid sequence is an excellent cell membrane-penetrating peptide, this construct can very efficiently traverse the cell membrane of eukaryotic cells. This makes it possible to efficiently introduce the foreign substance of interest from outside a eukaryotic cell into the cytoplasm of the cell.

### CITATION LIST

### Patent Literature

Patent Literature 1: Japanese Patent No. 3854995
Patent Literature 2: WO 2011/013700

### Non Patent Literature

Non Patent Literature 1: Journal of Biological Chemistry, Vol. 281, No. 35 (2006), Pages 25223 to 25230
Non Patent Literature 2: Proceedings of the National Academy of Sciences of the United States of America (PNAS), Vol. 98, No. 11 (2001), Pages 6336 to 6341

### SUMMARY OF INVENTION

### Technical Problem

In recent years, there has been increasing interest in peptides that exhibit an excellent membrane permeability, and from, inter alia, a therapeutic perspective, there is desire for the development of art for the introduction of foreign substances into target cells at better efficiencies than heretofore available.

The present invention was therefore created in order to respond to this demand, and takes as an object the introduction of a method of efficiently introducing a foreign substance of interest from outside a eukaryotic cell into at least the cytoplasm of the cell. An additional object of the present invention is to provide a construct of a foreign substance of interest with a carrier peptide fragment that brings about the efficient introduction of the foreign substance from outside a eukaryotic cell into the cytoplasm of the cell.

### Solution to Problem

In order to realize these objects, the present inventors sought an amino acid sequence that would have an excellent cell membrane permeability with regard to transfer from outside a cell into the cell interior and that could be advantageously used as a carrier peptide (carrier peptide fragment). As a result, the present inventors discovered that humanin (HN), which originates with mitochondria present in human cells and is composed of the amino acid sequence given in SEQ ID NO: 1, has an excellent cell membrane permeability with regard to transfer from outside a cell into the cell interior and can be advantageously used as a carrier peptide fragment. Humanin is a peptide composed of the 24 amino acid residues reported in Non Patent Literature 2. To date, it is known that humanin has the function, inter alia, of inhibiting neuronal cell death and is a peptide secreted from within the cell to the cell exterior. However, it was not known that humanin transfers at high efficiencies from outside the cell into the cell interior, and this characteristic was discovered for the first time by the present inventors.

That is, the herein disclosed method is a method for introducing (transferring) a foreign substance of interest from the outside (i.e., on the outer side of the cell membrane) of a eukaryotic cell (in particular, various animal cells lacking a cell wall, as represented by human cells and nonhuman mammalian cells) into at least the cytoplasm of the cell. The herein disclosed foreign substance introduction method comprises:
(1) a step of preparing a foreign substance introduction construct that has a carrier peptide fragment comprising the following amino acid sequence:
MAPRGFSCLLLLTSEIDLPVKRRA (SEQ ID NO: 1)
   and a foreign substance of interest bonded to the N-terminal side and/or the C-terminal side of the carrier peptide fragment;
(2) a step of supplying the foreign substance introduction construct into a sample that contains a target eukaryotic cell; and
(3) a step of incubating the sample into which the foreign substance introduction construct has been supplied, to thereby introduce the construct into the eukaryotic cell in the sample.

Here, “foreign substance” refers to inorganic compounds and organic compounds that can be bonded, either directly or indirectly through a suitable linker, to the N-terminal side or C-terminal side of the carrier peptide fragment, and that have the molecular size and chemical characteristics that support introduction or transfer into the interior of a eukaryotic cell.

In accordance with the foreign substance introduction method having the indicated constitution, a foreign substance of interest can be very efficiently introduced from outside a eukaryotic cell (outer side of the cell membrane) across the cell membrane into the cytoplasm by supplying (i.e., adding to living eukaryotic cells)—to a sample containing target eukaryotic cells (typically a culture containing these cells)—a foreign substance introduction construct fabricated by bonding the foreign substance of interest (typically an organic compound such as a polypeptide, nucleic acid, dye, or drug), directly or indirectly via a suitable linker, to the N-terminal side and/or C-terminal side of the aforementioned carrier peptide fragment.

In a preferred aspect of the herein disclosed foreign substance introduction method, the foreign substance is characteristically any organic compound selected from the group consisting of polypeptides, nucleic acids, dyes, and drugs. Constructs fabricated to contain these types of organic compounds are efficiently introduced into target cells.

Here, “polypeptide” denotes a polymer having a structure in which a plurality of amino acids are bonded by the peptide bond. The polypeptide is not limited by the number of peptide bonds (i.e., the number of amino acid residues). Thus, polypeptides encompass compounds generally known as peptides, which have approximately from 10 to fewer than 300 amino acid residues, and compounds generally known as proteins (typically macromolecular compounds composed of at least 300 amino acid residues). Polypeptides are not rigorously distinguished from proteins in this field. In the present Description, polypeptide is used to comprehensively refer to polymers composed of a plurality of amino acid residues (including oligomers).

In addition, “nucleic acid” refers to nucleotide polymers and encompasses DNA and RNA. This “nucleic acid” is not limited with respect to the number of bases.

In a preferred aspect of the herein disclosed foreign substance introduction method, the foreign substance is a mature polypeptide originating from any biological species or a precursor polypeptide therefor, and the foreign substance introduction construct is then a synthetic polypeptide having the amino acid sequence of the instant carrier peptide fragment plus an amino acid sequence that corresponds to the mature polypeptide or precursor polypeptide therefor as the foreign substance.

This constitution enables the efficient introduction into the target eukaryotic cell of the synthetic polypeptide having the amino acid sequence of the carrier peptide fragment plus an amino acid sequence of a mature polypeptide or precursor polypeptide therefor.

In another preferred aspect of the herein disclosed foreign substance introduction method, the amino acid sequence that corresponds to the mature polypeptide or precursor polypeptide therefor used as the foreign substance is located at the N-terminal side of the carrier peptide fragment.

This constitution enables an even more efficient introduction into the target eukaryotic cell of the amino acid sequence of the mature polypeptide or precursor polypeptide therefor.

In another preferred aspect of the herein disclosed foreign substance introduction method, the eukaryotic cell targeted for the introduction of the foreign substance introduction construct is a human cell or a cell from a nonhuman mammal.

This constitution enables the efficient introduction of a foreign substance into the cytoplasm of human cells or cells from a nonhuman mammal.

In order to realize the aforementioned objects, the present invention also provides an artificially fabricated foreign substance introduction construct for the purpose of introducing (transferring) a foreign substance of interest from the outside (i.e., on the outer side of the cell membrane) of a eukaryotic cell (in particular, various animal cells lacking a cell wall, as represented by human cells and nonhuman mammalian cells) into at least the cytoplasm of the cell.

The herein disclosed foreign substance introduction construct thus comprises a carrier peptide fragment comprising MAPRGFSCLLLLTSEIDLPVKRRA (SEQ ID NO: 1) and said foreign substance of interest bonded to the N-terminal side and/or the C-terminal side of this carrier peptide fragment.

A foreign substance of interest can be efficiently introduced into a target eukaryotic cell by carrying out the hereinabove disclosed foreign substance introduction method using this construct.

In a preferred aspect of the herein disclosed foreign substance introduction construct, as described above the foreign substance is any organic compound selected from the group consisting of polypeptides, nucleic acids, dyes, and drugs.

In addition, preferably this foreign substance is a mature polypeptide originating from any biological species, or a precursor polypeptide therefor, and the foreign substance introduction construct is a synthetic polypeptide having the amino sequence of the carrier peptide fragment plus an amino acid sequence that corresponds to the mature polypeptide or precursor polypeptide therefor as the foreign substance.

This amino acid sequence that corresponds to the mature polypeptide or precursor polypeptide therefor as the foreign substance is more preferably located on the N-terminal side of the carrier peptide fragment.

### BRIEF DESCRIPTION OF DRAWINGS

FIG. 1 is a histogram that shows the relationship between fluorescent intensity and cell count, as obtained by flow cytometric analysis of the cells after culture for the following: a test (Example 1) in which sample 1, which had FAM and the amino acid sequence given by SEQ ID NO: 1, was added to a culture of HeLa cells; a test (Example 2) in which sample 2, which had FAM and the amino acid sequence given by SEQ ID NO: 2, was added to a culture of HeLa cells; a test (Example 3) in which FAM was added to a culture of HeLa cells; and a test (Example 4) in which only DMSO was added to a culture of HeLa cells. The X-axis shows the fluorescent intensity and the Y-axis shows the cell count.

### DESCRIPTION OF EMBODIMENTS

Preferred embodiments of the present disclosure are described in the following. Matters required for the implementation of the present art, but which are not particularly described in the present Description (for example, general matters related to methods for the chemical synthesis of peptides, cell culture techniques, and the preparation of compositions that contain peptides and/or nucleic acids as components) can be understood as design matters for the individual skilled in the art based on the conventional art in fields such as cell engineering, physiology, medicine, pharmaceutical science, organic chemistry, biochemistry, genetic engineering, protein engineering, molecular biology, genetics, and so forth.

The present art can be implemented based on the contents disclosed in the present Description and the common general technical knowledge in the pertinent fields. In the description that follows, the amino acids are represented, depending on the circumstances, by the single letter codes (except that three letter codes are used in the sequence listings) stipulated by the nomenclature for amino acids given in the IUPAC-IUB guidelines. Unless specifically indicated otherwise, in the present Description “amino acid residue” is a term that encompasses the N-terminal amino acid and the C-terminal amino acid of a peptide chain.

In the present Description, “synthetic peptide” refers to a peptide fragment for which the peptide chain thereof does not exist by itself in an independent and stable manner in nature, but rather is produced by artificial chemical synthesis or biosynthesis (i.e., production based on genetic engineering) and can exist in a stable manner in a prescribed composition. Herein, “peptide” is a term that indicates an amino acid polymer having a plurality of peptide bonds, and there is no limitation with regard to the number of amino acid residues.

For the amino acid sequences described in the present Description, the left side is always the N-terminal side and the right side always indicates the C-terminal side.

The herein disclosed foreign substance introduction construct has a carrier peptide fragment comprising MAPRGFSCLLLLTSEIDLPVKRRA (SEQ ID NO: 1) and the foreign substance of interest bonded to the N-terminal side and/or C-terminal side of this carrier peptide fragment.

The herein disclosed “carrier peptide fragment” is a sequence prescribed (identified) by the amino acid sequence given by SEQ ID NO: 1 and is an amino acid sequence that exhibits a cell membrane-penetrating capability for eukaryotic cells.

The amino acid sequence given by SEQ ID NO: 1 is the full length amino acid sequence of humanin (HN), which originates with the mitochondria present in human cells, and is an amino acid sequence composed of a total of 24 amino acid residues. It is already known that humanin exhibits an inhibitory effect on neuronal cell death (Non Patent Literature 2).

The amino acid sequence given by SEQ ID NO: 1 is a carrier peptide fragment that has an excellent cell membrane permeability with regard to introduction from outside a cell into the cell interior, and as a consequence a foreign substance introduction construct having this carrier peptide fragment and a foreign substance of interest bonded to the N-terminal side and/or the C-terminal side of this carrier peptide fragment, is introduced at higher efficiencies from outside a eukaryotic cell into at least the cytoplasm of the cell.

The herein disclosed “carrier peptide fragment” typically has the same amino acid sequence as the amino acid sequence given by SEQ ID NO: 1, but, insofar as the cell membrane permeability is not impaired, it encompasses sequences provided by modification of the amino acid sequence with SEQ ID NO: 1. These “modified sequences” are amino acid sequences (modified amino acid sequences) formed by the substitution, deletion, and/or addition (insertion) of one amino acid residue or a plurality (typically two or three) of amino acid residues. Such slightly modified sequences can be readily used by the individual skilled in the art based on the herein disclosed information and as a consequence are encompassed by the “carrier peptide fragment” as a herein disclosed technical concept.

The modified sequences in the present Description can be exemplified by sequences produced by a so-called conservative amino acid replacement in which 1, 2, or 3 amino acid residues are conservatively replaced. Typical examples of conservative substitutions are, for example, sequences in which a basic amino acid residue is substituted by a different basic amino acid residue (for example, exchange between a lysine residue and an arginine residue), and sequences in which a hydrophobic amino acid residue is substituted by a different hydrophobic amino acid residue (for example, exchange among the leucine residue, isoleucine residue, and valine residue).

The foreign substance introduction construct can be designed constructed by bonding (linking) a desired foreign substance, either directly or indirectly via a suitable linker, to the N-terminal side and/or the C-terminal side of the carrier fragment.

There are no particular limitations on the linker, and it may be a peptide linker or a nonpeptide linker. While not constituting a particular limitation, the amino acid sequence constituting a peptide linker preferably is an amino acid sequence that does not produce steric hindrance and is flexible. The peptide linker can be, for example, a linker that contains one or two or more amino acid residues selected from glycine, alanine, serine, and so forth and that is composed of not more than 10 amino acid residues (more preferably from one to five, for example, one, two, three, four, or five amino acid residues). β-alanine may be used for this linker. While not constituting a particular limitation, for example, an alkyl linker, polyethylene glycol (PEG) linker, aminohexanoyl spacer, and so forth may be used for the nonpeptide linker.

The foreign substance is typically an organic compound such as a polypeptide, nucleic acid, dye, or drug.

The foreign substance can be, for example, a polypeptide. When the foreign substance is a polypeptide, the target foreign substance introduction construct can be fabricated by designing a peptide chain that contains the amino acid sequence constituting this polypeptide and the amino acid sequence constituting the carrier peptide fragment, and by carrying out the biosynthesis or chemical synthesis of this peptide chain. In addition, foreign substance introduction constructs can be fabricated by the direct or indirect bonding to the N-terminal side and/or C-terminal side of the carrier peptide fragment, using various heretofore known chemical methods, of a nucleic acid, e.g., various DNAs and RNAs, a dye (for example, various fluorescent dye compounds, e.g., FAM, FITC), or an organic compound that functions as a drug (for example, antitumor agents, including nucleic acid-type antitumor agents, e.g., 5-fluorouracil (5FU), antivirals such as azidothymidine (AZT), and so forth).

While not being a particular limitation, the function possessed by the foreign substance can be, for example, promotion of the induction of stem cell differentiation (stem cell differentiation induction activity), inhibition of the proliferation of tumor cells (antitumor activity), inhibition of the proliferation of virus-infected cells (antiviral activity), and so forth.

There are no particular limitations on the number of foreign substances bonded to the carrier peptide fragment in the foreign substance introduction construct. Thus, one or more foreign substances may be bonded to one carrier peptide fragment. While not being a particular limitation, for example, a polypeptide, nucleic acid, drug, etc., may be bonded to the N-terminal side of one carrier peptide fragment and a dye may be bonded to the C-terminal side thereof. The bonding of a dye to the carrier peptide fragment facilitates evaluation of the efficiency of introduction into the eukaryotic cell of the foreign substance introduction construct as well as evaluation of the localization within the cell, and is thus preferred.

When the foreign substance is a polypeptide, there are no particular limitations on the polypeptide (amino acid sequence) that may be used. For example, a polypeptide that has a relatively large number of amino acid residues, such as a polypeptide or protein that has approximately 100 to 1,000 amino acid residues, can be used as the foreign substance.

The total number of amino acid resides constituting the synthetic peptide that is fabricated as the foreign substance introduction construct is typically at least several to several tens (for example, 10), and is suitably not more than 1,000, preferably not more than 600, still more preferably not more than 500, and particularly preferably not more than 300 (for example, to 300). Polypeptides of this length are easy to synthesize (biosynthesis, chemical synthesis) and easy to use.

The foreign substance is preferably the mature form of, or a precursor for (including proforms and pre-proforms), a polypeptide involved in a function such as the development, differentiation, growth, malignant transformation, homeostasis, regulation of metabolism, and so forth of various cells and tissues (organs). In addition, through the introduction into a cell of a polypeptide for which the function is not yet known, the herein disclosed foreign substance introduction method can also be used to elucidate the function within a cell (within biological tissue) of the polypeptide.

For example, when the eukaryotic cell that is the target of foreign substance introduction is a human or other mammalian stem cell, it will be advantageous to use the mature form of, or a precursor for, a polypeptide having any of various biological activities involved with the induction of stem cell differentiation. Here, “stem cell” encompasses somatic stem cells, embryonic stem cells, and induced pluripotent stem cells (iPS cells in the following). When the eukaryotic cell that is the target of foreign substance introduction is a cancer cell (tumor cell), it will be advantageous to use various polypeptides involved in the induction of apoptosis of cancer cells (tumor cells). Or, in this case it will be advantageous to use a polypeptide that can prevent cancer cells (tumor cells) from suppressing the function of the immune surveillance system. When the eukaryotic cell that is the target of introduction is a bacterially infected cell or a virally infected cell, it will be advantageous to use various polypeptides involved with the induction of apoptosis of the infected cells, and/or polypeptides that can inhibit the growth of the bacteria or virus in the infected cells, and/or polypeptides that can inhibit a broadening of the bacterial or viral infection from the infected cells.

Just as for the carrier peptide fragment, the polypeptide serving as the foreign substance may include, insofar as its function is preserved, a modified amino acid sequence formed by the substitution, deletion, and/or addition (insertion) of one or several amino acid residues.

The foreign substance introduction construct is preferably a foreign substance introduction construct in which at least one amino acid residue has been converted to the amide. The structural stability (for example, protease resistance) of the foreign substance introduction construct in the cytoplasm and nucleus can be improved when a carboxyl group of the amino acid residues (typically the C-terminal amino acid residue of the peptide chain) is converted to the amide. In addition, the hydrophilicity of such a construct is enhanced, and as a consequence the water solubility of such a construct can be improved.

For example, when the foreign substance is bonded to the N-terminal side of the carrier peptide fragment, the C-terminal amino acid residue of the carrier peptide fragment is preferably converted to the amide. In addition, when, for example, the foreign substance is a polypeptide and this polypeptide is bonded to the C-terminal side of the carrier peptide fragment, the C-terminal amino acid residue of the polypeptide is preferably converted to the amide.

A foreign substance introduction construct having a relatively short peptide chain (including the polypeptide constituting the foreign substance, the carrier peptide fragment, and the peptide linker) can be readily produced based on general chemical synthesis methods. For example, a heretofore known solid-phase synthesis method or liquid-phase synthesis method may be used. A solid-phase synthesis method using Boc (t-butyloxycarbonyl) or Fmoc (9-fluorenylmethoxycarbonyl) as the amino group protection group is preferred. Thus, the aforementioned peptide chain having the desired amino acid sequence and modified moieties (e.g., C-terminal amidation) can be synthesized by solid-phase synthesis using a commercial peptide synthesizer. Only a portion of the peptide chain may be synthesized by this method, for example, only the carrier peptide fragment, or a peptide chain containing the carrier peptide fragment and a peptide linker moiety may be synthesized by this method.

Alternatively, production may be carried out by biosynthesis of the peptide moiety based on genetic engineering methods. Thus, a polynucleotide (typically DNA) having a nucleotide sequence (containing the ATG start codon) encoding the desired amino acid sequence is synthesized. A recombinant vector having a gene expression construct comprising the synthesized polynucleotide (DNA) and various control elements (including a promoter, ribosome binding site, terminator, enhancer, and various cis elements that control the expression level) for bringing about the expression of the amino acid sequence in a host cell is constructed in correspondence to the host cell.

Using ordinary methods, this recombinant vector is introduced into a prescribed host cell (for example, yeast, insect cells, plant cells), and the host cell, or tissue or a specimen containing these cells, is cultured under prescribed conditions. The target peptide can thereby be produced in the cells. The peptide moiety is isolated from the host cell (from the medium when it has been secreted), and the target peptide moiety can be obtained by carrying out refolding, purification, and so forth, as necessary.

With regard to the method for constructing the recombinant vector, the method for introducing the constructed recombinant vector into a host cell, and so forth, methods already being used in this field may be directly used, and their detailed description has been omitted since these methods as such are not a particular characteristic feature of the present art.

For example, a fusion protein expression system can be used in order to bring about the efficient production of large amounts in a host cell. Thus, a gene (DNA) encoding the amino acid sequence of the target polypeptide is chemically synthesized, and the synthetic gene is introduced into the appropriate site in a suitable fusion protein expression vector (for example, the pET series available from Novagen, or a GST (glutathione S-transferase) fusion protein expression vector, such as the pGEX series available from Amersham Bioscience). A host cell (typically E. coli) is transformed with this vector. The resulting transformant is cultured to produce the target fusion protein. This protein is then extracted and purified. The obtained purified fusion protein is cleaved with a prescribed enzyme (protease), and the liberated target peptide fragment (i.e., the designed artificial polypeptide) is recovered by a method such as affinity chromatography. A desired foreign substance introduction construct (artificial polypeptide) can be produced using such a heretofore known fusion protein expression system (For example, the GST/His system provided by Amersham Bioscience can be used.).

Alternatively, a target polypeptide can be synthesized in vitro using a so-called cell-free protein synthesis system, by constructing a template DNA (i.e., a synthetic gene fragment that contains a nucleotide sequence encoding the amino acid sequence of the peptide moiety of the foreign substance introduction construct) for use in the cell-free protein synthesis system and by using the various compounds (ATP, RNA polymerase, amino acids, and so forth) required for synthesis of the peptide moiety. For example, the reports by Shimizu et al. (Shimizu et al., Nature Biotechnology, 19, 751-755 (2001)) and Madin et al. (Madin et al., Proc. Natl. Acad. Sci. USA, 97(2), 559-564 (2000)) are references for cell-free protein synthesis systems. At the time of the filing of the instant patent application, numerous enterprises were already performing contract polypeptide production based on the technology described in these reports and cell-free protein synthesis kits based on the technology described in these reports were commercially available (for example, kits can be acquired from CellFree Sciences Co., Ltd. in Japan).

The single-stranded or double-stranded polynucleotide containing the nucleotide sequence encoding the peptide moiety of the foreign substance introduction construct, and/or containing the nucleotide sequence complementary to this sequence, can be readily produced (synthesized) using heretofore known methods. Thus, the nucleotide sequence corresponding to this amino acid sequence can be readily determined and provided by selecting the codons corresponding to the individual amino acid residues comprising the designed amino acid sequence. Once the nucleotide sequence has been determined, the polynucleotide (single-stranded) corresponding to the desired nucleotide sequence can be readily obtained using, for example, a DNA synthesizer. Using the obtained single-stranded DNA as a template, the target double-stranded DNA can be obtained using various enzymatic synthesis means (typically PCR). The polynucleotide may be in the form of DNA or in the form of RNA (e.g., mRNA). The DNA can be provided as the double strand or single strand. When provided as the single strand, it may be the coding strand (sense strand) or may be the noncoding strand (antisense strand) having the complementary sequence thereto.

The thusly obtained polynucleotide can be used as material for constructing a recombinant gene (expression cassette) for peptide production in various host cells or using a cell-free protein synthesis system, as described in the preceding.

The foreign substance introduction construct can be favorably used as an effective component in compositions that are applied based on the function of the foreign substance. The foreign substance introduction construct may take the form of a salt insofar as the function of the foreign substance is not impaired. For example, use can be made of the acid-addition salt that can be obtained using a common method by the addition reaction of a commonly used inorganic acid or organic acid. The “foreign substance introduction construct” described in the present Description and in the claims thus encompasses these salt forms.

The foreign substance introduction construct can be provided in the form of a composition that can contain, in addition to the foreign substance introduction construct functioning as the effective component, any of various medically (pharmaceutically) acceptable carriers in accordance with the form of use.

A carrier as generally used in peptide drugs as, for example, a diluent, excipient, and so forth, is preferred for this carrier. This carrier can differ as appropriate in correspondence to the application and form of the foreign substance introduction construct, but is typically water, a physiological buffer solution, or any of various organic solvents. In addition, this carrier can be an aqueous solution of an alcohol (e.g., ethanol) at a suitable concentration, or can be glycerol or a nondrying oil such as olive oil, or may be a liposome. In addition, secondary components that can be incorporated in the drug composition can be exemplified by various fillers, expanders, binders, moisturizers, surfactants, dyes, fragrances, and so forth.

There are no particular limitations on the form of the composition. Typical forms can be exemplified by liquids, suspensions, emulsions, aerosols, foams, granules, powders, tablets, capsules, and ointments. In addition, for use as, inter alia, an injectable, a lyophilized material or granulated material can also be made in order to support the preparation of a drug solution by dissolution in, for example, physiological saline or a suitable buffer solution (for example, PBS) immediately prior to use.

The processes as such for the preparation of various drug formulations (compositions) using the foreign substance introduction construct (main component) and various carriers (secondary components) as materials may be based on heretofore known methods, and a detailed description of these formulation methods as such is omitted because they are not a characteristic feature of the present art. For example, Comprehensive Medicinal Chemistry, edited by Corwin Hansch, Pergamon Press (1990) is a source of detailed information with regard to formulations.

Also provided is a method, using the herein disclosed foreign substance introduction construct (composition), for introducing the foreign substance introduction construct within an organism (in vivo) or outside an organism (in vitro). In general terms, this method comprises the following steps (1) to (3):
(1) a step of preparing a foreign substance introduction construct having a carrier peptide fragment comprising the amino acid sequence given by SEQ ID NO: 1 and having the foreign substance of interest bonded to the N-terminal side and/or the C-terminal side of the carrier peptide fragment;
(2) a step of supplying this foreign substance introduction construct to a sample that contains target eukaryotic cells; and
(3) a step of incubating the sample to which the foreign substance introduction construct has been supplied, in order to introduce the construct into the eukaryotic cells in the sample.

With regard to in vivo, the “eukaryotic cells” here encompass, for example, various tissues, viscera, organs, blood, and lymph. With regard to in vitro, the “eukaryotic cells” here encompass, for example, various cell aggregates, tissues, viscera, organs, blood, and lymph removed from an organism, as well as cell lines.

For in vivo, compositions containing the hereinabove disclosed construct can be used in accordance with the methods and use amounts that correspond to the form of the composition and the goals therefor. For example, precisely the desired amount can be administered as a liquid to a diseased region (for example, malignant tumor tissue, virally infected tissue, inflamed tissue) of a patient (i.e., an organism) by intravenous, intramuscular, subcutaneous, intradermal, or intraperitoneal injection. Alternatively, a solid form, e.g., a tablet, or a gel or aqueous jelly, e.g., an ointment, can be administered directly to a prescribed tissue (i.e., for example, a diseased region, e.g., a tissue or organ that contains, e.g., tumor cells, virally infected cells, or inflamed cells). Alternatively, a solid form, e.g., a tablet, can be administered orally. In the case of oral administration, the use is preferred of an encapsulating or protective (coating) material in order to prevent degradation by digestive enzymes in the digestive tract.

Alternatively, for eukaryotic cells that are cultured outside an organism (in vitro), a suitable amount of the herein disclosed composition (i.e., a suitable amount of the foreign substance introduction construct) may be supplied at least once to the culture medium for the target eukaryotic cells. The amount per supply and number of times of supply are not particularly limited because they will vary depending on conditions such as, for example, the species of eukaryotic cell being cultured, cell density (cell density at the start of culture), number of subcultures, culture conditions, and type of culture medium. For example, a single addition or a plurality of additions, i.e., twice or more, is preferably made so as to provide a carrier peptide fragment concentration in the culture medium within the range of approximately at least 0.05 μM to not more than 100 μM, for example, within the range of at least 0.5 μM to not more than 50 μM, and, for example, within the range of at least 1 μM to not more than 20 μM.

An example of an in vitro method of introduction is given in the examples below.

There are no particular limitations on methods for evaluating the efficiency of introduction of the foreign substance introduction construct. For example, when a dye (typically a fluorescent dye compound) is bonded to this construct, the efficiency of introduction into eukaryotic cells can be evaluated using microscopic observations (for example, fluorescence microscopic observations) or flow cytometry. The efficiency of construct introduction can also be evaluated by an immunochemical method (e.g., western blot or immune cell staining) that uses an antibody that specifically recognizes the peptide moiety of the construct.

Several examples relating to the present art are described in the following, but this should not be construed as limiting the present art to what is shown in these examples.

<Fabrication of Foreign Substance Introduction Constructs>

The two synthetic peptides (peptide 1 and peptide 2) shown in Table 1 were prepared. Peptide 1 is a carrier peptide fragment composed of the amino acid sequence given by the herein disclosed SEQ ID NO: 1. Peptide 2 is an amino acid sequence, given by SEQ ID NO: 2, known as a nucleolar localization signal (NoLS) of the LIM kinase 2 present in human endothelial cells, and is the carrier peptide fragment disclosed in Patent Literature 2.

Peptide 1 and peptide 2 were both synthesized by carrying out solid-phase synthesis (Fmoc method) using a commercial peptide synthesizer and following the manual provided therewith. Peptide 2 was synthesized with the carboxyl group (—COOH) of the C-terminal amino acid residue being converted to the amide (—CONH₂).

The procedure as such for using the peptide synthesizer is not a characteristic feature of the present art, and a detailed explanation thereof has therefore been omitted.

TABLE 1

| peptide No. | sequence | SEQ ID NO: |
|---|---|---|
| 1 | MAPRGFSCLLLLTSEIDLPVKRRA | 1 |
| 2 | KKRTLRKNDRKKR | 2 |

A foreign substance introduction construct provided with peptide 1 (also referred to as “sample 1”) and a foreign substance introduction construct provided with peptide 2 (also referred to as “sample 2”) were then prepared by directly bonding, based on a standard method, the fluorescent dye FAM (C2141207: 5(6)-carboxyfluorescein, molecular weight=376.3, excitation wavelength=495 nm, fluorescence wavelength=520 nm) as the foreign substance to the amino acid residue on the N-terminal side of peptide 1 and peptide 2, respectively. Sample 1 and sample 2 were each diluted with DMSO to prepare a sample solution 1 having a sample 1 concentration of 2 mM and a sample solution 2 having a sample 2 concentration of 2 mM.

<Evaluation of the Cell Membrane Permeability of Sample 1 and Sample 2>

The cell membrane permeability of sample 1 and sample 2 was evaluated using HeLa cells (an established cell line derived from human cervical cancer cells) as the eukaryotic cells. As shown in Table 2, Example 1 indicates a test in which the sample solution 1 was added to a HeLa cell culture; Example 2 indicates a test in which the sample solution 2 was added to a HeLa cell culture; Example 3 indicates a test in which a DMSO-diluted FAM solution was added to a HeLa cell culture; and Example 4 indicates a test in which only DMSO was added to a HeLa cell culture.

TABLE 2

| | addition |
|---|---|
| Example 1 | FAM-MAPRGFSCLLLLTSEIDLPVKRRA-COOH |
| Example 2 | FAM-KKRTLRKNDRKKR-CONH₂ |
| Example 3 | FAM |
| Example 4 | none (only DMSO) |

### Example 1

HeLa cells were cultured on DMEM (Dulbecco's modified Eagle's medium (Cat. No. 043-30085, from FUJIFILM Wako Pure Chemical Corporation)) containing 10% FBS (fetal bovine serum), which is a general culture medium.

The HeLa cells attached to the culture plate were washed with PBS, a 0.25% trypsin/EDTA solution was then added, and incubation was carried out for 3 minutes at 37° C. After this incubation, the aforementioned DMEM containing 10% FBS was added and the trypsin was inactivated, and the cells were then sedimented by carrying out centrifugal separation for 5 minutes at 150×g. The supernatant produced by the centrifugal separation was removed, and the aforementioned DMEM containing 10% FBS was added to the sediment (cell pellet) to prepare a cell suspension containing approximately 1×10⁵ cells/mL. The cells were seeded (approximately 2×10⁵ cells/well) by the addition of 2 mL of this cell suspension to the wells of a commercial 6-well plate (Iwaki). The cells were attached to the well bottom by culture for 3 hours at 37° C. under the condition of 5% CO₂. In order to perform the evaluation of the cell membrane permeability in triplicate, the cells were seeded to three wells for the test of Example 1 and the following procedure was performed on each well.

The 2 mM sample solution 1 was then diluted with the aforementioned DMEM containing 10% FBS to prepare a sample solution 1 in which the sample 1 concentration was 20 μM. After the aforementioned 3-hour culture, 1 mL of the culture supernatant was removed from the well, followed by the addition to the well of 1 mL of the 20 μM sample solution 1 (i.e., the concentration of sample 1 in the culture medium in the well was brought to 10 μM and the DMSO concentration was brought to 0.5%). The cells were incubated for 20 hours at 37° C. under the condition of 5% CO₂. After this 20-hour incubation, the culture supernatant was removed from the well and the cells in the well were washed twice with 1 mL PBS. 200 μL of a 0.25% trypsin/EDTA solution was then added to the well and incubation was carried out for 3 minutes at 37° C. After this incubation, the trypsin was inactivated by the addition of 400 μL of the aforementioned DMEM containing 10% FBS to the well, and the cells were subsequently recovered by transferring the cell suspension in the well to a tube. The well was then washed by the further addition of 600 μL PBS to the well. The cells that had remained in the well were recovered to the tube by transferring the PBS in the well to the tube. Centrifugal separation was run on this tube for 5 minutes using conditions of 4° C. and 210×g. After the centrifugal separation, the supernatant was removed, the sediment (cell pellet) was suspended (washed) with 1 mL of PBS, and centrifugal separation was carried out using the same conditions as before. After this procedure had been performed twice, the supernatant was removed to obtain cells (cell pellet) that had been cultured on a sample 1-containing culture medium.

The cell membrane permeability of sample 1 was evaluated using the resulting cells (cell pellet) and a flow cytometer. An On-Chip Flow Cytometer (On-chip Biotechnologies Co., Ltd.) was used for the flow cytometer.

For this analysis, the cell pellet obtained as described above was suspended in 50 μL PBS. To this suspension was added 50 μL of Sample Buffer 2× for use with the indicated flow cytometer to prepare a cell suspension for submission to analysis.

Gating was performed using this flow cytometer based on forward scatter (FSC) and side scatter (SSC); the gating was set for the cell population that was the target of the analysis; and the fluorescent intensity was measured for the cell population within this gating. The analysis was performed so this cell population reached at least 5,000 or more. The fluorescence detector FL2 (optimal detection wavelength around 543 nm) on the indicated flow cytometer, which could detect the fluorescence wavelength of FAM, was used to measure the fluorescent intensity. The measurement results were analyzed using “FlowJo (registered trademark)” (Tree Star Inc.) commercial analysis software to obtain the mean fluorescent intensity (MFI) for the cell population that was the measurement target.

### Example 2

This was carried out as in Example 1, except that the sample solution 2 was used in place of the sample solution 1.

### Example 3

This was carried out as in Example 1, but using the DMSO-diluted FAM solution in place of the sample solution 1. A FAM solution concentration was used that provided the same concentrations as the sample 1 solution (i.e., a concentration was used that brought the FAM concentration in the culture medium in the well to 10 μM and the DMSO concentration to 0.5%).

### Example 4

This was carried out as in Example 1, but using DMSO for the sample solution 1.

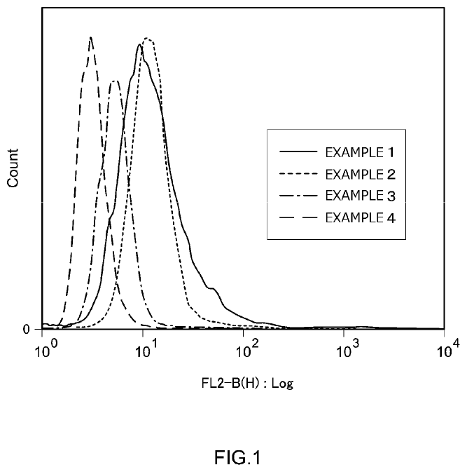

The results obtained in Examples 1 to 4 are given in Table 3 and FIG. 1. In Table 3, the mean value of the triplicate MFI's is given for each example. For each example, FIG. 1 provides a histogram of the flow cytometry of the triplicate test example that was nearest to the mean value of the MFI.

TABLE 3

| | Example 1 | Example 2 | Example 3 | Example 4 |
|---|---|---|---|---|
| MFI | 33.7 | 15.5 | 6 | 3.4 |

As shown in FIG. 1, for Example 1, which concerned the addition of the foreign substance introduction construct 1 that was provided with peptide 1 and a fluorescent dye (FAM), and for Example 2, which concerned the addition of the foreign substance introduction construct 2 that was provided with peptide 2 and a fluorescent dye (FAM), it was found that, based on the histogram shifting to the right on the X-axis, the fluorescent dye (FAM) functioning as the foreign substance was more efficiently introduced into the cytoplasm of the HeLa cells by peptide 1 and peptide 2 than in Example 3, in which only FAM was added. In addition, comparing Example 1 and Example 2, the histogram for Example 1 was shifted more to the right on the X-axis than was the histogram for Example 2. Thus, it was determined that, in comparison to the foreign substance introduction construct provided with peptide 2, the foreign substance introduction construct provided with peptide 1 had a higher cell membrane permeability and could introduce the foreign substance more efficiently from outside the cell into the cell interior. This efficiency (cell membrane permeability), when compared using the MFI given in Table 3, was found to be approximately twice as high for Example 1 as for Example 2.

While the detailed data are not provided, investigations by the present inventors have determined that not only in the case of the fluorescent dye, but also for the use of polypeptides, nucleic acids, and drugs as the foreign substance, such foreign substances are efficiently introduced from the cell exterior across the cell membrane and into the cytoplasm.

As is made clear from the preceding, the herein disclosed art provides, as a particularly preferred embodiment of the foreign substance introduction method, a method of introducing a foreign substance of interest from the outside of a eukaryotic cell into at least the cell cytoplasm of the cell, said method being characterized by the use of a carrier peptide fragment comprising SEQ ID NO: 1. This carrier peptide fragment is favorable for the purpose of introducing a foreign substance of interest into the cytoplasm of a cell.

While specific examples of the herein disclosed art have been described in detail above, these are only illustrations and are not limitations on the claims. The art described in the claims encompasses various modifications and alterations of the specific examples provided above as illustrations.

### INDUSTRIAL APPLICABILITY

The herein disclosed art provides an artificially fabricated construct for introducing a foreign substance of interest from the outside of a eukaryotic cell (particularly cell wall-free cells from various animals as represented by humans and other mammals) into at least the cytoplasm of the cell. Through the use of this construct, a foreign substance of interest can be effectively introduced into a target cell and cells can be obtained into which the foreign substance has been introduced, as can biological tissue, e.g., organs, that contain cells that contain the foreign substance. In addition, therapeutic agents against diseases can be provided through the use of this construct.

1.

### (Sequence Listing Free Text)

SEQ ID NO: 2 synthetic peptide

1. A method for the introduction of a foreign substance of interest from the outside of a eukaryotic cell into at least the cytoplasm of the cell, the method comprising:
(1) a step of preparing a foreign substance introduction construct that has a carrier peptide fragment comprising the following amino acid sequence:
(SEQ ID NO: 1)
MAPRGFSCLLLLTSEIDLPVKRRA
and
the foreign substance of interest bonded to the N-terminal side and/or the C-terminal side of the carrier peptide fragment;
(2) a step of supplying the foreign substance introduction construct into a sample that contains a target eukaryotic cell; and
(3) a step of incubating the sample into which the foreign substance introduction construct has been supplied, to thereby introduce the construct into the eukaryotic cell in the sample.

2. The method according to claim 1, wherein the foreign substance is any organic compound selected from the group consisting of polypeptides, nucleic acids, dyes, and drugs.

3. The method according to claim 2, wherein the foreign substance is a mature polypeptide originating from any biological species, or a precursor polypeptide therefor, and the foreign substance introduction construct is a synthetic polypeptide having the amino sequence of the carrier peptide fragment and an amino acid sequence that corresponds to the mature polypeptide or precursor polypeptide therefor as the foreign substance.

4. The method according to claim 3, wherein the amino acid sequence that corresponds to the mature polypeptide or precursor polypeptide therefor used as the foreign substance is located at the N-terminal side of the carrier peptide fragment.

5. The method according to claim 1, wherein the eukaryotic cell that is the target for the introduction of the foreign substance introduction construct is a human cell or a cell from a nonhuman mammal.

6. A foreign substance introduction construct prepared in order to introduce a foreign substance of interest from outside a eukaryotic cell into at least the cytoplasm of the cell, the foreign substance introduction construct comprising:
a carrier peptide fragment comprising the following amino acid sequence:
(SEQ ID NO: 1)
MAPRGFSCLLLLTSEIDLPVKRRA
and
the foreign substance of interest bonded to the N-terminal side and/or the C-terminal side of the carrier peptide fragment.

7. The construct according to claim 6, wherein the foreign substance is any organic compound selected from the group consisting of polypeptides, nucleic acids, dyes, and drugs.

8. The construct according to claim 7, wherein the foreign substance is a mature polypeptide originating from any biological species, or a precursor polypeptide therefor, and the construct is a synthetic polypeptide having the amino sequence for the carrier peptide fragment and an amino acid sequence that corresponds to the mature polypeptide or precursor polypeptide therefor as the foreign substance.

9. The construct according to claim 8, wherein the amino acid sequence that corresponds to the mature polypeptide or precursor polypeptide therefor used as the foreign substance is located at the N-terminal side of the carrier peptide fragment.


SEQUENCE LISTING (2 sequences)

SEQ ID NO: 1 - protein, 24 aa, Homo sapiens
MAPRGFSCLLLLTSEIDLPVKRRA

SEQ ID NO: 2 - protein, 13 aa, Artificial Sequence
  synthetic peptide
KKRTLRKNDRKKR